(19) United States
(12) Patent Application Publication
(10) Pub. No.: US 2016/0136275 A1
(43) Pub. Date: May 19, 2016

(54) OPHTHALMIC COMPOSITION

(71) Applicant: ROHTO PHARMACEUTICAL CO., LTD., Osaka (JP)

(72) Inventors: Takayuki MIYANO, Osaka (JP); Takahiro KUROSE, Osaka (JP)

(21) Appl. No.: 15/007,274

(22) Filed: Jan. 27, 2016

Related U.S. Application Data
(63) Continuation of application No. 13/775,631, filed on Feb. 25, 2013.
(60) Provisional application No. 61/638,750, filed on Apr. 26, 2012.

(30) Foreign Application Priority Data
Feb. 27, 2012 (JP) 2012-040729

(51) Int. Cl. A61K 47/02; A61K 9/00; A61K 31/121

(52) U.S. Cl. CPC A61K 47/02; A61K 31/121; A61K 9/0048

(57) ABSTRACT

An ophthalmic composition comprising geranylgeranylacetone and a phosphate buffering agent has an advantage that the loss of the geranylgeranylacetone content during long-term storage is very little. This is because of reduced adsorption of geranylgeranylacetone to a wall of an ophthalmic container. The ophthalmic composition comprising geranylgeranylacetone and a phosphate buffering agent also has an advantage that adsorption of geranylgeranylacetone to a contact lens is little. Further, the ophthalmic composition comprising geranylgeranylacetone and a phosphate buffering agent hardly becomes white turbid even when stored at low temperature.

### TECHNICAL FIELD

The present invention relates to an ophthalmic composition comprising geranylgeranylacetone.

### BACKGROUND ART

Teprenone (Eisai Co., Ltd.) is a mixture of (5E,9E,13E)-geranylgeranylacetone (hereinafter sometimes referred to as “all-trans form”) and (5Z,9E,13E)-geranylgeranylacetone (hereinafter sometimes referred to as “5Z-mono-cis form”) at a weight ratio of 3:2. Teprenone is widely used as an oral therapeutic agent for gastric ulcer.

The use of teprenone in the ophthalmic field has been suggested. For example, Patent Literature 1 teaches the use of teprenone as an active ingredient of a prophylactic or therapeutic agent for dry eye, eye strain, or eye dryness.

Patent Literature 2 discloses a clear eye drop consisting of teprenone, a phospholipid, a synthetic surfactant, and water.

However, the stability of geranylgeranylacetone in the ophthalmic compositions described in Patent Literature 1 and 2 is not practically sufficient.

Generally, in order to improve the thermal and light stabilities of an active ingredient in an ophthalmic composition, a borate buffering agent is used (Patent Literature 3 to 6).

### CITATION LIST

### Patent Literature

Patent Literature 1: JP-8-133967 A

Patent Literature 2: JP-2000-319170 A

Patent Literature 3: Patent No. 2929274

Patent Literature 4: Patent No. 3146218

Patent Literature 5: JP-2006-151969 A

Patent Literature 6: JP-2008-94780 A

### SUMMARY OF INVENTION

### Technical Problem

An object of the present invention is to provide an ophthalmic composition comprising geranylgeranylacetone having a practically sufficient stability.

### Solution to Problem

The inventors conducted extensive research in order to solve the above problem and unexpectedly found that the addition of a phosphate buffering agent to an ophthalmic composition comprising geranylgeranylacetone (hereinafter sometimes referred to as “GGA”) improves the thermal and light stabilities of GGA and reduces white turbidity of the ophthalmic composition stored at low temperature. The inventors also found that the addition of a phosphate buffering agent to an ophthalmic composition being held by an ophthalmic container and comprising GGA effectively reduces adsorption of GGA to the wall of the ophthalmic container and a contact lens.

The present invention has been completed based on the above findings and provides an ophthalmic composition as described below.

(1) An ophthalmic composition comprising geranylgeranylacetone and a phosphate buffering agent.

(2) The ophthalmic composition according to the above (1), whose pH is from 6 to 8.

(3) The ophthalmic composition according to the above (1) or (2), wherein the phosphate buffering agent concentration expressed in terms of a corresponding anhydride is 0.001 to 10% by weight relative to the total amount of the composition.

(4) The ophthalmic composition according to any of the above (1) to (3), wherein the phosphate buffering agent is at least one selected from the group consisting of phosphoric acid and an alkali metal phosphate.

(5) The ophthalmic composition according to any of the above (1) to (4), wherein the geranylgeranylacetone content is 0.00001 to 10% by weight relative to the total amount of the composition.

(6) A method for reducing the loss of the geranylgeranylacetone content of an ophthalmic composition, the method comprising the step of adding a phosphate buffering agent to an ophthalmic composition being held by an ophthalmic container and comprising geranylgeranylacetone, thereby reducing the loss of the geranylgeranylacetone content of the ophthalmic composition.

(7) A method for reducing white turbidity due to geranylgeranylacetone during storage at low temperature, the method comprising the step of adding a phosphate buffering agent to an ophthalmic composition comprising geranylgeranylacetone, thereby reducing white turbidity due to geranylgeranylacetone during storage at low temperature.

(8) A method for reducing adsorption of geranylgeranylacetone to a contact lens, the method comprising the step of adding a phosphate buffering agent to an ophthalmic composition comprising geranylgeranylacetone, thereby reducing adsorption of geranylgeranylacetone to a contact lens.

(9) A method for stabilizing geranylgeranylacetone, the method comprising the step of adding a phosphate buffering agent to an ophthalmic composition comprising geranylgeranylacetone, thereby stabilizing geranylgeranylacetone.

(10) A method for reducing adsorption of geranylgeranylacetone to a wall of an ophthalmic container, the method comprising the step of adding a phosphate buffering agent to an ophthalmic composition being held by an ophthalmic container and comprising geranylgeranylacetone, thereby reducing adsorption of geranylgeranylacetone to a wall of the ophthalmic container.

(11) Use of a combination of geranylgeranylacetone and a phosphate buffering agent for the production of an ophthalmic composition.

(12) Use, as an ophthalmic composition, of a composition comprising geranylgeranylacetone and a phosphate buffering agent.

### Advantageous Effects of Invention

Generally, the GGA content of an ophthalmic composition tends to be reduced during storage. In contrast, the ophthalmic composition of the present invention has an advantage that the loss of the GGA content during long-term storage is very little. The loss of the GGA content of the ophthalmic composition of the present invention varies depending on the material of an ophthalmic container and hence a container material of some kind allows an added phosphate buffering agent to reduce adsorption of GGA to the inner wall of an ophthalmic container. The ophthalmic composition of the present invention also has an advantage that the GGA in the composition is very stable to light and heat.

Generally, an ophthalmic composition comprising GGA tends to become white turbid when stored at low temperature. Consequently, during commercial distribution to or during storage in cold areas, such an ophthalmic composition becomes white turbid, which reduces its commercial value. In contrast, the ophthalmic composition of the present invention hardly becomes white turbid even when stored at low temperature. Therefore, the ophthalmic composition of the present invention can be commercially distributed to any area and thus its commercial value is high.

Generally, GGA tends to be adsorbed to a contact lens. Adsorption of a component of an ophthalmic composition to a contact lens reduces the effect given by the component and wearing the contact lens contaminated by the adsorption may cause blurred vision or damage the eye. These problems will not occur with the use of the ophthalmic composition of the present invention.

### DESCRIPTION OF EMBODIMENTS

The present invention will be described in detail below. The ophthalmic composition of the present invention is an ophthalmic composition comprising GGA and a phosphate buffering agent.

### Geranylgeranylacetone

### (1) Types of Geometric Isomers

GGA has eight geometric isomers. Specifically, the eight geometric isomers are:
(5E,9E,13E)-6,10,14,18-tetramethyl-5,9,13,17-nonadecatetrae n-2-one (5E,9E,13E GGA) (all-trans form),
(5Z,9E,13E)-6,10,14,18-tetramethyl-5,9,13,17-nonadecatetrae n-2-one (5Z,9E,13E GGA) (5Z-mono-cis form),
(5Z,9Z,13E)-6,10,14,18-tetramethyl-5,9,13,17-nonadecatetrae n-2-one (5Z,9Z,13E GGA) (13E-mono-trans form),
(5Z,9Z,13Z)-6,10,14,18-tetramethyl-5,9,13,17-nonadecatetrae n-2-one (5Z,9Z,13Z GGA) (all-cis form),
(5E,9Z,13E)-6,10,14,18-tetramethyl-5,9,13,17-nonadecatetrae n-2-one (5E,9Z,13E GGA) (9Z-mono-cis form),
(5E,9Z,13Z)-6,10,14,18-tetramethyl-5,9,13,17-nonadecatetrae n-2-one (5E,9Z,13Z GGA) (5E-mono-trans form),
(5E,9E,13Z)-6,10,14,18-tetramethyl-5,9,13,17-nonadecatetrae n-2-one (5E,9E,13Z GGA) (13Z-mono-cis form),
and
(5Z, 9E,13Z) -6,10,14,18-tetramethyl-5,9,13,17-nonadecatetrae n-2-one (SZ,9E,13Z GGA) (9E-mono-trans form).

These GGAs can be used alone or in any combination of two or more thereof according to the present invention. In cases where two or more of the GGAs are combined, the mixing ratio is not particularly limited.

Among the above GGAs, preferred are the all-trans form, the mono-cis forms (especially the 5Z-mono-cis form) and a mixture of the all-trans form and one of the mono-cis forms.

In cases where the GGA of the present invention is a mixture of the all-trans form and one of the mono-cis forms (especially the 5Z-mono-cis form), the all-trans form content of the mixture is preferably 80% by weight or more, more preferably 82% by weight or more, further more preferably 84% by weight or more, further more preferably 86% by weight or more, further more preferably 88% by weight or more, further more preferably 90% by weight or more, further more preferably 92% by weight or more, further more preferably 94% by weight or more, further more preferably 96% by weight, further more preferably 98% by weight or more. Especially preferably, the GGA consists of the all-trans form. When the all-trans form is in the above ranges, white turbidity at low temperature is reduced.

Also preferred GGA is a mixture of the all-trans form and one of the mono-cis forms (especially the 5Z-mono-cis form) with a very high mono-cis form (especially the 5Z-mono-cis form) ratio.

### (2) All-Trans Form and 5Z-mono-cis Form

(5E, 9E,13E) -geranylgeranylacetone (the all-trans form) is a compound represented by the following structural formula:

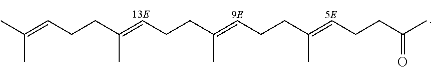 [SMILES: CC(=O)CC/C=C(\C)CC/C=C(\C)CC/C=C(\C)CCC=C(C)C]

The all-trans form can be purchased from, for example, Rionlon Development Co., Ltd.

The all-trans form can also be obtained through separating the all-trans form and the 5Z-mono-cis form of a marketed teprenone (Eisai Co., Ltd., Wako Pure Chemical Industries, Ltd., Yoshindo Inc., etc.) by, for example, silica gel chromatography using a mobile phase of n-hexane/ethyl acetate (9:1). The separation of the all-trans form and the 5Z-mono-cis form of a marketed teprenone can also be commissioned to, for example, KNC Laboratories Co., Ltd.

(5Z,9E,13E)-geranylgeranylacetone (the 5Z-mono-cis form) can also be obtained by the separation from a marketed teprenone.

The 5Z-mono-cis form is a compound represented by the following structural formula:

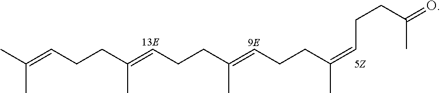 [SMILES: CC(=O)CC/C=C(/C)CC/C=C(\C)CC/C=C(\C)CCC=C(C)C]

The all-trans form can also be synthesized in accordance with a method described in, for example, Bull. Korean Chem. Soc., 2009, Vol. 30, No. 9, 215-217. This literature describes, for example, the method shown by the following synthesis scheme:

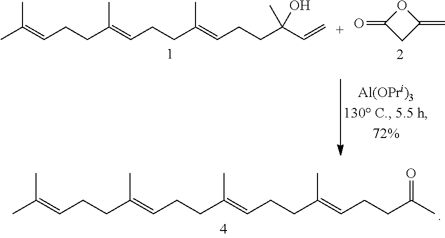

Specifically, in the above reaction formula, geranyllinalool 1 is mixed with Compound 2 and aluminum isopropoxide, and the mixture is gradually heated to 130° C. to allow the reaction to occur. After the completion of the reaction, the residue Compound 2 is removed and the reaction mixture is diluted with 5% sodium carbonate so that the residue aluminum propoxide is quenched. In this way, the all-trans form can be obtained. The obtained all-trans form is subsequently purified by, for example, silica gel chromatography using dichloromethane as an eluent.

### (3) Mixtures of All-Trans Form and 5Z-mono-cis Form

Mixtures of the all-trans form and the 5Z-mono-cis form can be obtained by adding the all-trans form or the 5Z-mono-cis form to a marketed teprenone.

### GGA Content

The GGA content is preferably 0.00001% by weight or more, more preferably 0.0001% by weight or more, further more preferably 0.001% by weight or more, relative to the total amount of the composition. The GGA content may be 0.01% by weight or more, 0.1% by weight or more, or 1% by weight or more. The GGA in the above ranges is sufficient to exert its pharmacological action.

The GGA content of the ophthalmic composition is preferably 10% by weight or less, more preferably 5% by weight or less, further more preferably 3% by weight or less, relative to the total amount of the composition. The ophthalmic composition comprising GGA in the above ranges allows clearer vision and hardly causes blurred vision.

The GGA content of the ophthalmic composition is, for example, about 0.00001 to 10% by weight, about 0.00001 to 5% by weight, about 0.00001 to 3% by weight, about 0.0001 to 10% by weight, about 0.0001 to 5% by weight, about 0.0001 to 3% by weight, about 0.001 to 10% by weight, about 0.001 to 5% by weight, about 0.001 to 3% by weight, about 0.01 to 10% by weight, about 0.01 to 5% by weight, about 0.01 to 3% by weight, about 0.1 to 10% by weight, about 0.1 to 5% by weight, about 0.1 to 3% by weight, about 1 to 10% by weight, about 1 to 5% by weight, or about 1 to 3% by weight, relative to the total amount of the composition.

### Phosphate Buffering Agent

Phosphate buffering agents can be used alone or in combination of two or more thereof.

The phosphate buffering agent is not particularly limited and examples thereof include phosphoric acid; alkali metal phosphates such as disodium hydrogen phosphate, sodium dihydrogen phosphate, trisodium phosphate, dipotassium hydrogen phosphate, potassium dihydrogen phosphate, and tripotassium phosphate; alkaline earth metal phosphates such as calcium phosphate, calcium hydrogen phosphate, calcium dihydrogen phosphate, monomagnesium phosphate, dimagnesium phosphate (magnesium hydrogen phosphate), and trimagnesium phosphate; and ammonium phosphates such as diammonium hydrogen phosphate and ammonium dihydrogen phosphate. The phosphate buffering agent may be an anhydride or hydrate.

Among the above, preferably at least one selected from the group consisting of phosphoric acid and alkali metal phosphates is used, and more preferably at least one selected from the group consisting of phosphoric acid and sodium phosphates is used.

Preferred combinations of phosphate buffering agents are, for example, a combination of phosphoric acid, disodium hydrogen phosphate, sodium dihydrogen phosphate, and trisodium phosphate; a combination of phosphoric acid, disodium hydrogen phosphate, and sodium dihydrogen phosphate; a combination of phosphoric acid, disodium hydrogen phosphate, and trisodium phosphate; a combination of phosphoric acid, sodium dihydrogen phosphate, and trisodium phosphate; a combination of disodium hydrogen phosphate, sodium dihydrogen phosphate, and trisodium phosphate; a combination of phosphoric acid and disodium hydrogen phosphate; a combination of phosphoric acid and sodium dihydrogen phosphate; a combination of phosphoric acid and trisodium phosphate; a combination of disodium hydrogen phosphate and sodium dihydrogen phosphate; a combination of disodium hydrogen phosphate and trisodium phosphate; and a combination of sodium dihydrogen phosphate and trisodium phosphate.

Among these, preferred are a combination of phosphoric acid, disodium hydrogen phosphate, and sodium dihydrogen phosphate; a combination of phosphoric acid and disodium hydrogen phosphate; a combination of phosphoric acid and sodium dihydrogen phosphate; and a combination of disodium hydrogen phosphate and sodium dihydrogen phosphate. More preferred is a combination of disodium hydrogen phosphate and sodium dihydrogen phosphate.

The phosphate buffering agent content expressed in terms of a corresponding anhydride is preferably 0.001% by weight or more, more preferably 0.005% by weight or more, further more preferably 0.01% by weight or more, further more preferably 0.05% by weight or more, relative to the total amount of the composition. The phosphate buffering agent in the above ranges is sufficient to exhibit the effects of stabilizing GGA, reducing white turbidity at low temperature, and reducing adsorption of GGA to a container wall or a contact lens.

The phosphate buffering agent content expressed in terms of a corresponding anhydride is preferably 10% by weight or less, more preferably 7% by weight or less, further more preferably 5% by weight or less, further more preferably 3% by weight or less, relative to the total amount of the composition. When GGA is in the above ranges, the ophthalmic composition exhibits reduced eye irritancy.

The phosphate buffering agent content expressed in terms of a corresponding anhydride is, for example, about 0.001 to 10% by weight, about 0.001 to 7% by weight, about 0.001 to 5% by weight, about 0.001 to 3% by weight, about 0.005 to 10% by weight, about 0.005 to 7% by weight, about 0.005 to 5% by weight, about 0.005 to 3% by weight, about 0.01 to 10% by weight, about 0.01 to 7% by weight, about 0.01 to 5% by weight, about 0.01 to 3% by weight, about 0.05 to 10% by weight, about 0.05 to 7% by weight, about 0.05 to 5% by weight, or about 0.05 to 3% by weight, relative to the total amount of the ophthalmic composition.

The phosphate buffering agent content expressed in terms of a corresponding anhydride is preferably 0.0005 parts by weight or more, more preferably 0.001 parts by weight or more, further more preferably 0.005 parts by weight or more, further more preferably 0.01 parts by weight or more, relative to 1 part by weight of GGA. The phosphate buffering agent in the above ranges is sufficient to exhibit the effects of stabilizing GGA, reducing white turbidity at low temperature, and reducing adsorption of GGA to a container wall or a contact lens.

The phosphate buffering agent content expressed in terms of a corresponding anhydride is preferably 5000 parts by weight or less, more preferably 1000 parts by weight or less, further more preferably 500 parts by weight or less, further more preferably 200 parts by weight or less, relative to 1 part by weight of GGA. When the phosphate buffering agent is in the above ranges, the ophthalmic composition exhibits reduced eye irritancy.

The phosphate buffering agent content expressed in terms of a corresponding anhydride is, for example, about 0.0005 to 5000 parts by weight, about 0.0005 to 1000 parts by weight, about 0.0005 to 500 parts by weight, about 0.0005 to 200 parts by weight, about 0.001 to 5000 parts by weight, about 0.001 to 1000 parts by weight, about 0.001 to 500 parts by weight, about 0.001 to 200 parts by weight, about 0.005 to 5000 parts by weight, about 0.005 to 1000 parts by weight, about 0.005 to 500 parts by weight, about 0.005 to 200 parts by weight, about 0.01 to 5000 parts by weight, about 0.01 to 1000 parts by weight, about 0.01 to 500 parts by weight, or about 0.01 to 200 parts by weight, relative to 1 part by weight of GGA.

### Preparations

The form of the ophthalmic composition may be a liquid, a fluid, a gel or a semi-solid. Generally, components in a liquid or fluid composition tend to be adsorbed to a container wall. Hence, the present invention is suitably applied to a liquid or fluid ophthalmic composition. In addition, GGA in an aqueous composition tends to be adsorbed to a container wall and thus the present invention is also suitably applied to an aqueous composition.

The type of the ophthalmic composition is not particularly limited. Examples thereof include an eye drop, an eye wash, a contact lens-wearing solution, a contact lens solution (e.g., a washing solution, a storage solution, a sterilizing solution, a multipurpose solution, a package solution, etc.), a preservative for a harvested ocular tissue (a cornea etc.) for transplantation, an irrigating solution for surgery, an ophthalmic ointment (e.g., a water-soluble ophthalmic ointment, an oil-soluble ophthalmic ointment, etc.), an intraocular injection (e.g., an intravitreal injection), etc. Among these, preferred are an eye drop, an eye wash, an ophthalmic ointment and an intraocular injection.

Preparation methods for an ophthalmic preparation are well known. An ophthalmic preparation can be prepared by mixing GGA with a pharmaceutically acceptable base or carrier, and as needed a pharmaceutically acceptable additive for an ophthalmic preparation and another active ingredient (a physiologically or pharmacologically active component).

### <Bases or Carriers>

Examples of the base or carrier include water; an aqueous solvent such as a polar solvent; a polyalcohol; a vegetable oil; and an oily base. Examples of the base or carrier for an intraocular injection include water for injection and physiological saline.

These bases or carriers can be used alone or in combination of two or more thereof.

### <Additives>

Examples of the additive include a surfactant, a flavor or cooling agent, an antiseptic, a bactericide or antibacterial agent, a pH adjusting agent, a tonicity agent, a chelating agent, another buffering agent, a stabilizer, an antioxidant, and a thickening agent. An intraocular injection may contain a solubilizing agent, a suspending agent, a tonicity agent, a buffering agent, a soothing agent, a stabilizer, and an antiseptic.

These additives can be used alone or in combination of two or more thereof.

The additives will be exemplified below.

Surfactants: for example, nonionic surfactants such as polyoxyethylene (hereinafter sometimes referred to as “POE”)-polyoxypropylene (hereinafter sometimes referred to as “POP”) block copolymers (e.g., poloxamer 407, poloxamer 235, poloxamer 188), ethylenediamine POE-POP block copolymer adducts (e.g., poloxamine), POE sorbitan fatty acid esters (e.g., polysorbate 20, polysorbate 60, polysorbate 80 (TO-10 etc.)), POE hydrogenated castor oils (e.g., POE (60) hydrogenated castor oil (HCO-60 etc.)), POE castor oils, POE alkyl ethers (e.g., polyoxyethylene (9) lauryl ether, polyoxyethylene (20) polyoxypropylene (4) cetyl ether), and polyoxyl stearate;
amphoteric surfactants such as glycine-type amphoteric surfactants (e.g., alkyl diaminoethyl glycine, alkyl polyaminoethyl glycine), betaine-type amphoteric surfactants (e.g., lauryldimethylaminoacetic betaine, imidazolinium betaine);
cationic surfactants such as alkyl quaternary ammonium salts (e.g., benzalkonium chloride, benzethonium chloride); etc.

The numbers in the parentheses represent the molar number of added POE or POP.

Flavors or cooling agents: for example, camphor, borneol, terpenes (these may be in the d-form, l-form, or dl-form); essential oils such as mentha water, eucalyptus oil, bergamot oil, anethole, eugenol, geraniol, menthol, limonene, mentha oil, peppermint oil, rose oil, etc.

Antiseptics, bactericides, or antibacterial agents: for example, polidronium chloride, alkyldiaminoethylglycine hydrochloride, sodium benzoate, ethanol, benzalkonium chloride, benzethonium chloride, chlorhexidine gluconate, chlorobutanol, sorbic acid, potassium sorbate, sodium dehydroacetate, methyl paraoxybenzoate, ethyl paraoxybenzoate, propyl paraoxybenzoate, butyl paraoxybenzoate, oxyquinoline sulfate, phenethyl alcohol, benzyl alcohol, biguanide compounds (in particular, polyhexamethylene biguanide or its hydrochloride etc.), Glokill (Rhodia Ltd.), etc.

pH adjusting agents: for example, hydrochloric acid, sodium hydroxide, potassium hydroxide, calcium hydroxide, magnesium hydroxide, triethanolamine, monoethanolamine, diisopropanolamine, sulfuric acid, phosphoric acid, etc.

Tonicity agents: for example, sodium bisulfite, sodium sulfite, potassium chloride, calcium chloride, sodium chloride, magnesium chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, glycerin, propylene glycol, etc.

Chelating agents: for example, ascorbic acid, edetic acid tetrasodium, sodium edetate, citric acid, etc.

Other buffering agents: for example, phosphate buffering agents; citrate buffering agents such as citric acid and sodium citrate; acetate buffering agents such as acetic acid, potassium acetate, and sodium acetate; carbonate buffering agents such as sodium bicarbonate and sodium carbonate; borate buffering agents such as boric acid and borax; amino acid buffering agents such as taurine, aspartic acid and its salts (e.g., potassium salts etc.), and ε-aminocaproic acid; etc.

These buffering agents can be added in an amount that does not affect the effect of the phosphate buffering agent.

Stabilizers: for example, trometamol, sodium formaldehyde sulfoxylate (rongalit), tocopherol, sodium pyrosulfite, monoethanolamine, aluminum monostearate, glyceryl monostearate, etc.

Antioxidants: for example, water-soluble antioxidants such as ascorbic acid, ascorbic acid derivatives (ascorbic acid-2-sulfate disodium salts, sodium ascorbate, ascorbic acid-2-magnesium phosphate, ascorbic acid-2-sodium phosphate, etc.), sodium bisulfite, sodium sulfite, sodium thiosulfate, etc.

The antioxidant may be a fat-soluble antioxidant. The addition of a fat-soluble antioxidant to the ophthalmic composition of the present invention reduces adsorption of the ophthalmic composition to a container wall, thereby further effectively reducing the loss of the GGA content of the composition. The addition of a fat-soluble antioxidant also reduces adsorption of GGA to a contact lens and improves the thermal and light stabilities of GGA.

Examples of the fat-soluble antioxidant include butyl group-containing phenols such as butylhydroxytoluene (BHT) and butylhydroxyanisole (BHA); nordihydroguaiaretic acid (NDGA); ascorbic acid esters such as ascorbyl palmitate, ascorbyl stearate, ascorbyl aminopropyl phosphate, ascorbyl tocopherol phosphate, ascorbic acid triphosphate, and ascorbyl palmitate phosphate; tocopherols such as α-tocopherol, β-tocopherol, γ-tocopherol, and δ-tocopherol; tocopherol derivatives such as tocopherol acetate, tocopherol nicotinate, and tocopherol succinate; gallic acid esters such as ethyl gallate, propyl gallate, octyl gallate, and dodecyl gallate; propyl gallate; 3-butyl-4-hydroxyquinolin-2-one; vegetable oils such as soybean oil, rapeseed oil, olive oil, and sesame oil; carotenoids such as lutein and astaxanthin; polyphenols such as anthocyanins, catechin, tannin, and curcumin; the vitamin A group including retinol, retinol esters (retinol acetate, retinol propionate, retinol butyrate, retinol octylate, retinol laurate, retinol stearate, retinol myristate, retinol oleate, retinol linolenate, retinol linoleate, retinol palmitate, etc.), retinal, retinal esters (retinal acetate, retinal propionate, retinal palmitate, etc.), retinoic acid, retinoic acid esters (methyl retinoate, ethyl retinoate, retinol retinoate, tocopheryl retinoate, etc.), dehydro forms of retinol, dehydro forms of retinal, dehydro forms of retinoic acid, provitamin A (α-carotene, β-carotene, γ-carotene, δ-carotene, lycopene, zeaxanthin, β-cryptoxanthin, echinenone, etc.), and vitamin A; CoQ10, etc. These compounds are marketed.

Among these, preferred are butyl group-containing phenols, NDGA, ascorbic acid esters, tocopherols, tocopherol derivatives, gallic acid esters, propyl gallate, and 3-butyl-4-hydroxyquinolin-2-one, vegetable oils, and the vitamin A group. Among these, preferred are butyl group-containing phenols, tocopherols, tocopherol derivatives, vegetable oils, and the vitamin A group, more preferred are butyl group-containing phenols, vegetable oils, retinol, and retinol esters, and further more preferred are BHT, BHA, sesame oil, and retinol palmitate.

These fat-soluble antioxidants can be used alone or in combination of two or more thereof.

The fat-soluble antioxidant content of the ophthalmic composition is preferably 0.00001% by weight or more, more preferably 0.00005% by weight or more, further more preferably 0.0001% by weight or more, further more preferably 0.0005% by weight or more, relative to the total amount of the ophthalmic composition. The fat-soluble antioxidant in the above ranges is sufficient to exhibit the effects of reducing adsorption of GGA to a container wall (thereby reducing the loss of the GGA content), reducing adsorption of GGA to a contact lens, and improving the thermal and light stabilities of GGA.

The fat-soluble antioxidant content of the ophthalmic composition is preferably 10% by weight or less, more preferably 5% by weight or less, further more preferably 2% by weight or less, further more preferably 1% by weight or less, relative to the total amount of the composition. When the fat-soluble antioxidant is in the above ranges, the ophthalmic composition exhibits reduced eye irritancy.

The fat-soluble antioxidant content of the ophthalmic composition is, for example, about 0.00001 to 10% by weight, about 0.00001 to 5% by weight, about 0.00001 to 2% by weight, about 0.00001 to 1% by weight, about 0.00005 to 10% by weight, about 0.00005 to 5% by weight, about 0.00005 to 2% by weight, about 0.00005 to 1% by weight, about 0.0001 to 10% by weight, about 0.0001 to 5% by weight, about 0.0001 to 2% by weight, about 0.0001 to 1% by weight, about 0.0005 to 10% by weight, about 0.0005 to 5% by weight, about 0.0005 to 2% by weight, or about 0.0005 to 1% by weight, relative to the total amount of the ophthalmic composition.

The fat-soluble antioxidant content of the ophthalmic composition is preferably 0.0001 parts by weight or more, more preferably 0.001 parts by weight or more, further more preferably 0.005 parts by weight or more, further more preferably 0.01 parts by weight or more, relative to 1 part by weight of GGA. The fat-soluble antioxidant in the above ranges is sufficient to exhibit the effects of reducing adsorption of GGA to a container wall (thereby reducing the loss of the GGA content), reducing adsorption of GGA to a contact lens, and improving the thermal and light stabilities of GGA.

The fat-soluble antioxidant content of the ophthalmic composition is preferably 100 parts by weight or less, more preferably 50 parts by weight or less, furthermore preferably 10 parts by weight or less, further more preferably 5 parts by weight or less, relative to 1 part by weight of GGA. When the fat-soluble antioxidant is in the above ranges, the ophthalmic composition exhibits reduced eye irritancy.

The fat-soluble antioxidant content of the ophthalmic composition is, for example, about 0.0001 to 100 parts by weight, about 0.0001 to 50 parts by weight, about 0.0001 to 10 parts by weight, about 0.0001 to 5 parts by weight, about 0.001 to 100 parts by weight, about 0.001 to 50 parts by weight, about 0.001 to 10 parts by weight, about 0.001 to 5 parts by weight, about 0.005 to 100 parts by weight, about 0.005 to 50 parts by weight, about 0.005 to 10 parts by weight, about 0.005 to 5 parts by weight, about 0.01 to 100 parts by weight, about 0.01 to 50 parts by weight, about 0.01 to 10 parts by weight, or about 0.01 to 5 parts by weight, relative to 1 part by weight of GGA.

Thickening agents: for example, guar gum; hydroxypropyl guar gum; high molecular cellulose compounds such as methylcellulose, ethylcellulose, hydroxypropyl methylcellulose, hydroxyethyl cellulose, and carboxymethyl cellulose sodium; gum arabic; karaya gum; xanthan gum; agar-agar; alginic acid; α-cyclodextrin; dextrin; dextran; heparin; heparinoid; heparin sulfate; heparan sulfate; hyaluronic acid; hyaluronates (sodium salts etc.); sodium chondroitin sulfate; starch; chitin and its derivatives; chitosan and its derivatives; carrageenan; sorbitol; high molecular polyvinyl compounds such as polyvinylpyrrolidone, polyvinyl alcohol, and polyvinyl methacrylate; carboxy vinyl polymers such as alkali metal polyacrylates (sodium salts, potassium salts, etc.), amine polyacrylates (monoethanolamine salts, diethanolamine salts, triethanolamine salts, etc.), and ammonium polyacrylates; casein; gelatin; collagen; pectin; elastin; ceramide; liquid paraffin; glycerin; polyethylene glycol; macrogol; polyethyleneimine alginates (sodium salts etc.); alginate esters (propylene glycol esters etc.); powdered tragacanth; triisopropanolamine; etc.

### <Other Pharmacologically or Physiologically Active Components>

Pharmacologically or physiologically active components other than GGA can be used alone or in combination of two or more thereof.

Examples of the pharmacologically or physiologically active components include prophylactic or therapeutic components for a retinal disease, nerve growth factors, decongestants, drugs for restoring extraocular muscle function, anti-inflammatory drugs or astringent drugs, antihistaminics or antiallergics, vitamins, amino acids, antibacterial drugs or bactericides, sugars, high molecular compounds, celluloses or their derivatives, local anesthetics, etc. These components will be exemplified below.

Prophylactic or therapeutic components for a retinal disease: for example, prostaglandin F2α derivatives such as prost drugs (latanoprost, travoprost, tafluprost, etc.), prostamide drugs (bimatoprost etc.) and prostone drugs (isopropyl unoprostone); sympatholytic drugs such as β-blockers (timolol maleate, gel-forming timolol, carteolol hydrochloride, gel-forming carteolol, etc.), β1-blockers (betaxolol hydrochloride etc.), αβ-blockers (levobunolol hydrochloride, nipradilol, bunazosin hydrochloride, etc.) and α2 blockers (brimonidine tartrate); parasympathomimetic drugs such as pilocarpine hydrochloride and distigmine bromide; sympathomimetic drugs such as epinephrine, epinephrine hydrogen tartrate and dipivefrin hydrochloride; carbonic anhydrase inhibitors such as dorzolamide hydrochloride and brinzolamide; specific inhibitors to ROCK (Rho-associated coiled coil forming protein kinase) such as SNJ-1656 and K-115; calcium antagonists such as lomerizine hydrochloride; EP2 agonists such as DE-117; adenosine A2a receptor agonists such as OPA-6566; therapeutic agents for age-related macular degeneration such as VEGF aptamers (pegaptanib sodium) and VEGF inhibitors (ranibizumab, bevacizumab); etc.

Nerve growth factors: for example, nerve growth factor (NGF), brain-derived nerve growth factor (BDNF), glial cell line-derived neurotrophic factor (GDNF), etc.

Since nutritional factors including nerve growth factors are contained in serum, it is possible to add serum from a patient to a preparation for the patient.

Decongestants: for example, α-adrenergic agonists such as epinephrine, epinephrine hydrochloride, ephedrine hydrochloride, oxymetazoline hydrochloride, tetrahydrozoline hydrochloride, naphazoline hydrochloride, phenylephrine hydrochloride, methylephedrine hydrochloride, epinephrine hydrogen tartrate, naphazoline nitrate, etc. These maybe in the d-form, l-form, or dl-form.

Drugs for restoring extraocular muscle function: for example, cholinesterase inhibitors having an active center similar to that of acetylcholine, such as neostigmine methylsulfate, tropicamide, helenien, atropine sulfate, etc.

Anti-inflammatory drugs or astringent drugs: for example, zinc sulfate, zinc lactate, allantoin, ε-aminocaproic acid, indomethacin, lysozyme chloride, silver nitrate, pranoprofen, azulene sulfonate sodium, dipotassium glycyrrhizinate, diammonium glycyrrhizinate, diclofenac sodium, bromfenac sodium, berberine chloride, berberine sulfate, etc.

Antihistaminics or antiallergics: for example, acitazanolast, diphenhydramine or its salts (hydrochloride etc.), chlorpheniramine maleate, ketotifen fumarate, levocabastine or its salts (hydrochloride etc.), amlexanox, ibudilast, tazanolast, tranilast, oxatomide, suplatast or its salts (tosilate etc.), sodium cromoglicate, pemirolast potassium, etc.

Vitamins: for example, retinol acetate, retinol palmitate, pyridoxine hydrochloride, flavin adenine dinucleotide sodium, pyridoxal phosphate, cyanocobalamin, panthenol, calcium pantothenate, sodium pantothenate, ascorbic acid, tocopherol acetate, tocopherol nicotinate, tocopherol succinate, tocopherol calcium succinate, ubiquinone derivatives, etc.

Amino acids: for example, aminoethylsulfonic acid (taurine), glutamic acid, creatinine, sodium aspartate, potassium aspartate, magnesium aspartate, magnesium potassium aspartate, sodium glutamate, magnesium glutamate, ε-aminocaproic acid, glycine, alanine, arginine, lysine, γ-aminobutyric acid, γ-aminovaleric acid, sodium chondroitin sulfate, etc. These may be in the d-form, l-form, or dl-form.

Antibacterial drugs or bactericides: for example, alkylpolyaminoethylglycine, chloramphenicol, sulfamethoxazole, sulfisoxazole, sulfamethoxazole sodium, sulfisoxazole diethanolamine, sulfisoxazole monoethanolamine, sulfisomezole sodium, sulfisomidine sodium, ofloxacin, norfloxacin, levofloxacin, lomefloxacin hydrochloride, acyclovir, etc.

Sugars: for example, monosaccharides, disaccharide, in particular, glucose, maltose, trehalose, sucrose, cyclodextrin, xylitol, sorbitol, mannitol, etc.

High molecular compounds: for example, alginic acid, sodium is alginate, dextrin, dextran, pectin, hyaluronic acid, chondroitin sulfate, (completely or partially saponified) polyvinyl alcohol, polyvinylpyrrolidone, carboxy vinyl polymers, macrogol, pharmaceutically acceptable salts thereof, etc.

Celluloses or their derivatives: for example, ethylcellulose, hydroxyethyl cellulose, hydroxypropyl cellulose, hydroxypropyl methylcellulose, methylcellulose, carboxymethyl cellulose, carboxymethylcellulose sodium, carboxyethyl cellulose, nitrocellulose, etc.

Local anesthetics: for example, chlorobutanol, procaine hydrochloride, lidocaine hydrochloride, etc.

pH

The pH of the ophthalmic preparation is preferably 4 or higher, more preferably 5.5 or higher, further more preferably 6 or higher, further more preferably 6.5 or higher. The preparation having a pH value in the above ranges is excellent in the thermal and light stabilities of GGA.

The pH of the ophthalmic preparation is preferably 9 or lower, more preferably 8.5 or lower, further more preferably 8 or lower, further more preferably 7.5 or lower. The ophthalmic preparation having a pH value in the above ranges exhibits reduced eye irritancy.

### Usage

The usage of the ophthalmic composition of the present invention varies depending on its dosage form and the route of administration is appropriately selected in accordance with the dosage form.

For example, when the composition of the present invention is an eye drop, the eye drop comprising GGA in the above concentration ranges is instilled, for example, about 1 to 5 times a day, preferably about 1 to 3 times a day, in an amount of about 1 to 2 drops each time.

When the composition of the present invention is an eye wash, eye washing is performed, for example, about 1 to 10 times a day, preferably about 1 to 5 times a day, each time using about to 20 mL of the eye wash comprising GGA in the above concentration ranges.

When the composition of the present invention is an ophthalmic ointment, the ophthalmic ointment comprising GGA in the above concentration ranges is applied to the eye, for example, about 1 to 5 times a day, preferably about 1 to 3 times a day, in an amount of about 0.001 to 5 g each time.

When the composition of the present invention is an intraocular injection, the intraocular injection comprising GGA in the above concentration ranges is injected, for example, about 1 to 3 times per day to 14 days, preferably once per day to 14 days, in an amount of about 0.005 to 1 mL each time.

When the composition of the present invention is a contact lens solution (a washing solution, a storage solution, a sterilizing solution, a multipurpose solution, package solution, etc.), a preservative for a harvested ocular tissue (a cornea etc.) for transplantation, or an irrigating solution for surgery, such a composition comprising GGA in the above concentration ranges is used in a usual dosage and regimen of such a type of preparation.

When the composition of the present invention is a sustained-release contact lens preparation, the contact lens comprising GGA in the above amount is replaced with a fresh one, for example, about 1 to 3 times per day to 14 days, preferably once per day to 14 days.

When the composition of the present invention is a sustained-release intraocular implant, about 1 to 14 days after the implantation of the implant comprising GGA in the above amount, a fresh one is implanted as needed.

The administration period varies depending on the type and stage of the disease, the age, weight, and sex of the patient, the route of administration, etc., and can be selected as appropriate, for example, from the range from about one day to 30 years. When the retinal protective action exhibited by the ophthalmic composition of the present invention suppresses the progress of a retinal disease, the administration can be further continued.

### Others

The present invention includes a method for reducing the loss of the GGA content of an ophthalmic composition, the method comprising adding a phosphate buffering agent to an ophthalmic composition being held by an ophthalmic container and comprising GGA,
a method for reducing adsorption of GGA to a wall of an ophthalmic container, the method comprising adding a phosphate buffering agent to an ophthalmic composition being held by an ophthalmic container and comprising GGA,
a method for reducing white turbidity due to GGA during storage at low temperature, the method comprising adding a phosphate buffering agent to an ophthalmic composition comprising GGA, a method for reducing adsorption of GGA to a contact lens, the method comprising adding a phosphate buffering agent to an ophthalmic composition comprising GGA, and
a method for stabilizing GGA, the method comprising adding a phosphate buffering agent to an ophthalmic composition comprising GGA.

In these methods of the present invention, the components, dosage, properties, dosage form, etc. of the ophthalmic composition are as described for the ophthalmic composition of the present invention.

The material of the ophthalmic container is not particularly limited as long as the material is usually used for an ophthalmic container. Examples of the ophthalmic container include an ophthalmic container whose surface in contact with the ophthalmic composition is at least partially or wholly made of at least one material selected from the group consisting of a polyolefin, an acrylic acid resin, a terephthalic acid ester, a 2,6-naphthalene dicarboxylic acid ester, a polycarbonate, a polymethylterpene, a fluorine resin, a polyvinyl chloride, a polyamide, an ABS resin, an AS resin, a polyacetal, a modified polyphenylene ether, a polyarylate, a polysulfone, a polyimide, a cellulose acetate, a hydrocarbon optionally substituted with a halogen atom, a polystyrene, a polybutylene succinate, an aluminum and a glass.

Examples of the polyolefin include polyethylenes (including high density polyethylene, low density polyethylene, ultra low densitypolyethylene, linear low densitypolyethylene, ultra high molecular weight polyethylene, etc.), polypropylenes (including isotactic polypropylene, syndiotactic polypropylene, atactic polypropylene, etc.), ethylene-propylene copolymers, etc.

Examples of the acrylic acid resin include acrylic acid esters such as methyl acrylate, methacrylic acid esters such as methyl methacrylate, cyclohexyl methacrylate and t-butyl cyclohexyl methacrylate, etc.

Examples of the terephthalic acid ester include polyethylene terephthalate, polytrimethylene terephthalate, polybutylene terephthalate, etc.

Examples of the 2,6-naphthalene dicarboxylic acid ester include polyethylene naphthalate, polybutylene naphthalate, etc.

Examples of the fluorine resin include fluorine-substituted polyethylenes (polytetrafluoroethylene, polychlorotrifluoroethylene, etc.), polyvinylidene fluoride, polyvinyl fluoride, perfluoroalkoxy fluorine resins, tetrafluoroethylene-hexafluoropropylene copolymers, ethylene-tetrafluoroethylene copolymers, ethylene-chlorotrifluoroethylene copolymers, etc.

Examples of the polyamide include nylon etc.

Examples of the polyacetal include polyacetals consisting of oxymethylene units, polyacetals containing oxyethylene units, etc.

Examples of the modified polyphenylene ether include polystyrene-modified polyphenylene ether etc.

Examples of the polyarylate include amorphous polyarylate etc.

Examples of the polyimide include aromatic polyimides such as the one obtained by polymerizing pyromellitic dianhydride and 4,4′-diaminodiphenyl ether.

Examples of the cellulose acetate include cellulose diacetate, cellulose triacetate, etc.

Examples of the hydrocarbon optionally substituted with a halogen atom include hydrocarbons such as methane, ethane, propane, butane, ethylene, propylene, 1-butene, 2-butene and 1,3-butadiene; hydrocarbons substituted with a fluorine atom; hydrocarbons substituted with a chlorine atom; hydrocarbons substituted with a bromine atom; hydrocarbons substituted with an iodine atom; etc.

### EXAMPLES

The present invention will be described in more detail below with reference to Examples, but the present invention is not limited thereto.

### (1) Preparation of Geranylgeranylacetone

Marketed teprenone (all-trans form:5Z-mono-cis form=6:4 (weight ratio)) (Wako Pure Chemical Industries, Ltd.) was purchased and the all-trans form was separated and purified by silica gel chromatography.

The above preparative purification was carried out using silica gel (PSQ60B, Fuji Silysia Chemical Ltd.) filled in a glass tube and a mobile phase of n-hexane/ethyl acetate (9:1). After the separation, each fraction was concentrated and dried under reduced pressure and the degree of purification and structure of the all-trans form were determined by GC and ¹H-NMR (solvent: deuterated chloroform; internal standard: tetramethylsilane) (about 20% yield).

### <GC Measurement Conditions>

Column: DB-1 (J&W Scientific, 0.53 mm×30 m, film thickness of 1.5 μm)
Column temperature: elevated at a rate of 5° C./minute from 200° C. to 300° C. (10 minutes)
Vaporizing chamber temperature: 280° C.
Detector temperature: 280° C.
Carrier gas: helium
Hydrogen pressure: 60 kPa
Air pressure: 50 kPa

Makeup gas pressure: 75 kPa (nitrogen gas)
Total flow: 41 mL/min
Column flow: 6.52 mL/min
Linear velocity: 58.3 cm/sec
Split ratio: 5:1
Injection volume: 1 μL of 0.1 g/100 mL sample (in ethanol)

### (2) Measurement Method for GGA Concentration

In accordance with the measurement conditions for the elution test described in PFSB/ELD Notification No. 0412007 “teprenone 100 mg/g fine granule”, the GGA concentration of each eye drop was determined from the area value of the 5Z-mono-cis form (Ac) and the area value of the all-trans form (At) using Japanese pharmacopoeia “teprenone reference standard (all-trans form:5Z-mono-cis form=about 6:4 (weight ratio), Pharmaceutical and Medical Device Regulatory Science Society of Japan)” or teprenone (Wako Pure Chemical Industries) as a reference standard under the HPLC measurement conditions described below. For the eye drop containing teprenone (all-trans form:5Z-mono-cis form=3:2 (weight ratio)), the GGA content was calculated by summing the amounts of the all-trans form and the 5Z-mono-cis form.

### <HPLC Measurement Conditions>

Detector: ultraviolet absorption spectrometer (measurement wavelength: 210 nm)
Column: YMC-Pack ODS-A (inner diameter: 4.6 mm, length: 15 cm, particle diameter: 3 μm)
Column temperature: 30° C.
Mobile phase: 90% acetonitrile solution
Flow rate: 1.2 to 1.3 mL/min (the 5Z-mono-cis form and the all-trans form are eluted in this order.)
Injection volume: 5 μL of 0.05 g/100 mL sample

### (3) Light Stability Test

Eye drops containing the marketed teprenone or GGA consisting of the all-trans form purified by the above method were prepared as follows. The constitutions of the eye drops are shown in Tables 1 and 2 below.

Specifically, to a surfactant (polysorbate 80, POE castor oil, etc.) warmed to 65° C., teprenone or the all-trans form, and optionally BHT, were added and dissolved under stirring in a hot water bath at 65° C. for 2 minutes. Water at 65° C. was added and each buffer was added under stirring to give a homogeneous solution. The pH and osmotic pressure were adjusted with hydrochloric acid and/or sodium hydroxide. This resulting solution was filtered through a membrane filter with a pore size of 0.2 μm (bottle top filter, Thermo Fisher Scientific) to give a clear sterile eye drop. Before the preparation of the sterile is eye drops, it was confirmed by HPLC described later that adsorption of GGA to instruments etc., which leads to the loss of the GGA content, did not occur during the preparation procedure.

A polyethylene terephthalate container (8 mL) (the container for Rohto Dryaid EX, Rohto Pharmaceutical) was completely filled with each of the prepared eye drops in an aseptic manner. Each eye drop was subjected to light irradiation under the following conditions. Teprenone or the all-trans form content in each sample was quantified immediately after the production and after the irradiation and the residual ratio (%) was calculated.
Irradiation equipment: LTL-200A-15WCD (Nagano Science) Light source: D-65 lamp
Total irradiation: 1,300,000 lx·h (4000 lx×325 hours)
Temperature and humidity: 25° C. and 60% RH
Direction of light irradiation: the light was irradiated from the top to the container left to stand in the upright position on the spinning disk of the equipment.

The results are shown in Tables 1 and 2.

### (4) Thermal Stability Test

Eye drops having the constitutions shown in Table 2 below were prepared and filtered in the same manner as in the preparation method described above. Each of the eye drops was filled into the polyethylene terephthalate container (8 mL) described above or a 10 mL clear glass container (Nichiden-Rika Glass) in an aseptic manner. For these eye drops, the stability test was performed by leaving the containers to stand in the upright position at 40° C., 50° C. or 60° C. for 10 days or 20 days. The teprenone or all-trans form content (g/100 mL) in each of the eye drops was quantified under the HPLC conditions described above immediately after the production and after being left to stand for a predetermined period of time, and the residual ratio (%) was calculated.

The results are shown in Table 2.

TABLE 1

| | | | | | Comparative |
|---|---|---|---|---|---|
| g/100 mL | | Example 1 | Example 2 | Example 3 | Example 1 |
| All-trans form | | 0.005 | 0.005 | 0.005 | 0.005 |
| Sodium dihydrogen | | 2.000 | 1.400 | 0.300 | — |
| phosphate dihydrate | | | | | |
| Disodium hydrogen | | 0.400 | 1.400 | 3.200 | — |
| phosphate | | | | | |
| dodecahydrate | | | | | |
| Boric acid | | — | — | — | 1.400 |
| Borax | | — | — | — | 0.300 |
| POE castor oil | | 0.002 | 0.002 | 0.002 | 0.002 |
| Polysorbate 80 | | 0.050 | 0.050 | 0.050 | 0.050 |
| Hydrochloric acid | | q.s. | q.s. | q.s. | q.s. |
| Sodium hydroxide | | q.s. | q.s. | q.s. | q.s. |
| Purified water | | q.s. | q.s. | q.s. | q.s. |
| pH | | 5.7 | 6.5 | 7.5 | 7.5 |
| Osmotic pressure | | 270 | 260 | 260 | 240 |
| mOsm | | | | | |
| Residual | 1,300,000 | 89.4 | 89.1 | 90.5 | 86.1 |
| ratio (%) | lx · h | | | | |

TABLE 2

| | | | | | | | | Comparative | Comparative |
|---|---|---|---|---|---|---|---|---|---|
| g/100 mL | | Example 4 | Example 5 | Example 6 | Example 7 | Example 8 | Example 9 | Example 2 | Example 3 |
| All-trans form | | 0.050 | 0.050 | 0.050 | — | — | — | 0.050 | — |
| All-trans form: | | — | — | — | 0.050 | 0.050 | 0.050 | — | 0.050 |
| 5Z-mono-cis form | | | | | | | | | |
| weight ratio(6:4) | | | | | | | | | |
| Sodium dihydrogen | | 2.000 | 1.400 | 0.300 | 2.000 | 1.400 | 0.300 | — | — |
| phosphate dihydrate | | | | | | | | | |
| Disodium hydrogen | | 0.400 | 1.400 | 3.200 | 0.400 | 1.400 | 3.200 | — | — |
| phosphate | | | | | | | | | |
| dodecahydrate | | | | | | | | | |
| Boric acid | | — | — | — | — | — | — | 1.400 | 1.400 |
| Borax | | — | — | — | — | — | — | 0.300 | 0.300 |
| POE castor oil | | 0.020 | 0.020 | 0.020 | 0.020 | 0.020 | 0.020 | 0.020 | 0.020 |
| Polysorbate 80 | | 0.500 | 0.500 | 0.500 | 0.500 | 0.500 | 0.500 | 0.500 | 0.500 |
| Hydrochloric acid | | q.s. | q.s. | q.s. | q.s. | q.s. | q.s. | q.s. | q.s. |
| Sodium hydroxide | | q.s. | q.s. | q.s. | q.s. | q.s. | q.s. | q.s. | q.s. |
| Purified water | | q.s. | q.s. | q.s. | q.s. | q.s. | q.s. | q.s. | q.s. |
| pH | | 5.7 | 6.5 | 7.5 | 5.7 | 6.5 | 7.5 | 7.5 | 7.5 |
| Osmotic pressure | | 270 | 260 | 260 | 270 | 260 | 260 | 240 | 240 |
| mOsm | | | | | | | | | |
| Storage conditions | | Residual ratio (%) | | | | | | | |
| 1,300,000 lx · h | PET | 91.3 | 91.9 | 92.8 | 90.6 | 91.8 | 91.9 | 89.0 | 88.4 |
| 40° C. 20 days | Glass | 99.9 | 99.7 | 100.1 | 99.5 | 99.9 | 99.6 | — | — |
| | PET | 98.4 | 98.0 | 99.5 | 97.3 | 98.3 | 99.1 | 96.7 | 97.1 |
| 50° C. 20 days | Glass | 97.9 | 99.0 | 99.3 | 96.5 | 97.5 | 98.9 | — | — |
| | PET | 91.9 | 94.3 | 99.6 | 90.2 | 94.6 | 99.4 | 72.4 | 83.4 |
| 60° C. 10 days | Glass | 97.9 | 101.3 | 101.7 | 96.0 | 100.5 | 98.3 | — | — |
| | PET | 84.8 | 90.7 | 97.2 | 85.6 | 91.8 | 96.9 | 72.5 | 81.5 |
| 60° C. 20 days | PET | 67.3 | 81.1 | 91.2 | 67.4 | 81.6 | 89.2 | 48.5 | 59.3 |

As is apparent from Tables 1 and 2, the thermal and light stabilities of GGA were higher in the eye drops containing the phosphate buffering agents than in the eye drops containing the borate buffering agents.

### (5) White Turbidity Reduction Test at Low Temperature

To a surfactant (polysorbate 80) warmed to 65° C., teprenone and the all-trans form were separately added and dissolved under stirring in a hot water bath at 65° C. for 2 minutes. Water at 65° C. was added and each buffer was added under stirring to give a homogeneous solution. The pH and osmotic pressure were adjusted with hydrochloric acid and/or sodium hydroxide. This resulting solution was filtered through a membrane filter with a pore size of 0.2 μm (bottle top filter, Thermo Fisher Scientific) to give an eye drop. Thus eye drops having the constitutions shown in Tables 3 and 4 were prepared.

A 10 mL clear glass container (Nichiden-Rika Glass) was completely filled with each of the eye drops (so that no air space remained). After sealing of the container, the eye drops were stored in the upright position at 4° C. Immediately after the preparation and after stored at 4° C. for three days or 14 days, 0.2 mL of each eye drop was transferred to wells of a 96-well plate (flat bottom, polystyrene) with a glass graduated pipette, and the absorbance was measured at 660 nm with a microplate reader (VersaMax, Molecular Devices) (temperature in the chamber: 20 to 25° C.). As referred to in JIS K0101 (Testing methods for industrial water, measurement of turbidity by light transmission), the absorbance at 660 nm of each sample was used as the indicator for white turbidity (the degree of turbidity).

The test procedure was carried out quickly. Before the test procedure was carried out, it was confirmed that the loss of the GGA content did not occur during the storage at 4° C. or the measurement of absorbance.

The results are shown in Tables 3 and 4.

TABLE 3

| | Example | Example | Example | Example | Example | Example | Comparative | Comparative |
|---|---|---|---|---|---|---|---|---|
| g/100 mL | 10 | 11 | 12 | 13 | 14 | 15 | Example 4 | Example 5 |
| All-trans form | 0.050 | 0.050 | 0.050 | — | — | — | 0.050 | — |
| All-trans form: | — | — | — | 0.050 | 0.050 | 0.050 | — | 0.050 |
| 5Z-mono-cis form | | | | | | | | |
| weight ratio (6:4) | | | | | | | | |
| Sodium dihydrogen | 2.000 | 1.400 | 0.300 | 2.000 | 1.400 | 0.300 | — | — |
| phosphate dihydrate | | | | | | | | |
| Disodium hydrogen | 0.400 | 1.400 | 3.200 | 0.400 | 1.400 | 3.200 | — | — |
| phosphate | | | | | | | | |
| dodecahydrate | | | | | | | | |
| Boric acid | — | — | — | — | — | — | 1.400 | 1.400 |
| Borax | — | — | — | — | — | — | 0.300 | 0.300 |
| Polysorbate 80 | 0.250 | 0.250 | 0.250 | 0.250 | 0.250 | 0.250 | 0.250 | 0.250 |
| Hydrochloric acid | q.s. | q.s. | q.s. | q.s. | q.s. | q.s. | q.s. | q.s. |
| Sodium hydroxide | q.s. | q.s. | q.s. | q.s. | q.s. | q.s. | q.s. | q.s. |
| Purified water | q.s. | q.s. | q.s. | q.s. | q.s. | q.s. | q.s. | q.s. |
| pH | 5.7 | 6.5 | 7.5 | 5.7 | 6.5 | 7.5 | 7.5 | 7.5 |
| Osmotic pressure | 270 | 260 | 260 | 270 | 260 | 260 | 240 | 240 |
| mOsm | | | | | | | | |
| 4° C. 3 days 660 nm | 0.0762 | 0.0734 | 0.0717 | 0.1250 | 0.1164 | 0.1056 | 0.1826 | 0.2302 |

The absorbance (660 nm) of water as a control was 0.0353.

TABLE 4

| | Example | Example | Example | Example | Comparative | Comparative |
|---|---|---|---|---|---|---|
| g/100 mL | 16 | 17 | 18 | 19 | Example 6 | Example 7 |
| All-trans form | 0.050 | 0.050 | — | — | 0.050 | — |
| All-trans form: | — | — | 0.050 | 0.050 | — | 0.050 |
| 5Z-mono-cis form | | | | | | |
| weight ratio (6:4) | | | | | | |
| Sodium dihydrogen | 2.000 | 0.300 | 2.000 | 0.300 | — | — |
| phosphate dihydrate | | | | | | |
| Disodium hydrogen | 0.400 | 3.200 | 0.400 | 3.200 | — | — |
| phosphate | | | | | | |
| dodecahydrate | | | | | | |
| Boric acid | — | — | — | — | 1.400 | 1.400 |
| Borax | — | — | — | — | 0.300 | 0.300 |
| Polysorbate 80 | 0.350 | 0.350 | 0.350 | 0.350 | 0.350 | 0.350 |
| Hydrochloric acid | q.s. | q.s. | q.s. | q.s. | q.s. | q.s. |
| Sodium hydroxide | q.s. | q.s. | q.s. | q.s. | q.s. | q.s. |
| Purified water | q.s. | q.s. | q.s. | q.s. | q.s. | q.s. |
| pH | 5.7 | 7.5 | 5.7 | 7.5 | 7.5 | 7.5 |
| Osmotic pressure | 270 | 260 | 270 | 260 | 240 | 240 |
| mOsm | | | | | | |
| 4° C. 14 days 660 nm | 0.0400 | 0.0417 | 0.0709 | 0.0690 | 0.1080 | 0.2358 |

The absorbance (660 nm) of water as a control was 0.0374.

As is apparent from Tables 3 and 4, white turbidity after storage at 4° C. was more reduced in the eye drops containing the phosphate buffering agents than in the eye drops containing the borate buffering agents.

As shown in Table 2, the residual ratio of GGA varied depending on the type of the container holding the eye drop. This explains that the use of a container of some type reduces adsorption of GGA to the container wall and such reduction allows the phosphate buffering agent to suppress the decrease in the residual ratio of GGA.

### (6) Test for Reduction in Adsorption to Contact Lenses

To a surfactant (polysorbate 80 and optionally POE castor oil) warmed to 65° C., teprenone or the all-trans form, and optionally BHT, were added and dissolved under stirring in a hot water bath at 65° C. for 2 minutes. Water at 65° C. was added and each buffer was added under stirring to give a homogeneous solution. The pH and osmotic pressure were adjusted with hydrochloric acid and/or sodium hydroxide. This resulting solution was filtered through a membrane filter with a pore size of 0.2 μm (bottle top filter, Thermo Fisher Scientific) to give an eye drop. Thus eye drops having the constitutions shown in Table 5 below were prepared. These eye drops were separately filled into a 4 mL clear glass container (Nichiden-Rika Glass).

One soft contact lens (hereinafter SCL): ACUVUE OASIS (Johnson & Johnson, approval number: 21800BZY10252000, base curve: 8.4 mm, diameter: 14.0 mm, power: −3.00 D) or ACUVUE ADVANCE (Johnson & Johnson, approval number: 21800BZY10251000, base curve: 8.3 mm, diameter: 14.0 mm, power: −3.00 D) was immersed in 4 mL of each eye drop (immersion solution) and left to stand in the upright position at 25° C., 60% RH for 8 hours, 14 hours or 24 hours. Each SCL had been initialized before use through immersion in 10 mL of physiological saline (Otsuka Normal Saline) overnight after being taken out from the package solution.

For 4 mL of the eye drop without immersion of SCL (blank solution), the same procedure as those for the eye drops with immersion of SCL (immersion solution) was performed. The amount of teprenone or the all-trans form was quantified by HPLC for each of the blank solution and the immersion solution, and the difference in the amounts between the blank solution and the immersion solution was used to calculate the amount of adsorption to SCL (μg/lens) (n=2).

Amount of adsorption (μg/lens)=[amount of teprenone or all-trans form in blank solution (g/100 mL)−amount of teprenone or all-trans form in immersion solution (g/100 mL)]/100×4×1000×1000

The results for ACUVUE OASIS and ACUVUE ADVANCE are shown in Tables 5 and 6, respectively.

TABLE 5

| | Example | Example | Example | Example | Comparative | Comparative |
|---|---|---|---|---|---|---|
| g/100 mL | 20 | 21 | 22 | 23 | Example 8 | Example 9 |
| All-trans form | 0.05 | 0.05 | — | — | 0.05 | — |
| All-trans form: | — | — | 0.05 | 0.05 | — | 0.05 |
| 5Z-mono-cis form | | | | | | |
| weight ratio (6:4) | | | | | | |
| Sodium dihydrogen | 2.00 | 0.30 | 2.00 | 0.30 | — | — |
| phosphate dihydrate | | | | | | |
| Disodium hydrogen | 0.40 | 3.20 | 0.40 | 3.20 | — | — |
| phosphate | | | | | | |
| dodecahydrate | | | | | | |
| Boric acid | — | — | — | — | 1.40 | 1.40 |
| Borax | — | — | — | — | 0.30 | 0.30 |
| Polysorbate 80 | 0.25 | 0.25 | 0.25 | 0.25 | 0.25 | 0.25 |
| Hydrochloric acid | q.s. | q.s. | q.s. | q.s. | q.s. | q.s. |
| Sodium hydroxide | q.s. | q.s. | q.s. | q.s. | q.s. | q.s. |
| Purified water | q.s. | q.s. | q.s. | q.s. | q.s. | q.s. |
| pH | 5.7 | 7.5 | 5.7 | 7.5 | 7.5 | 7.5 |
| Osmotic pressure | 270 | 260 | 270 | 260 | 240 | 240 |
| mOsm | | | | | | |
| Adsorption amount | 152.8 | 146.4 | 137.4 | 148.6 | 222.4 | 208.8 |
| (μg/lens) | | | | | | |
| Immersion | 132.8 | 156.3 | 143.1 | 159.1 | 219.7 | 207.8 |
| for 14 hours | | | | | | |

TABLE 6

| | | | | | Comparative | Comparative |
|---|---|---|---|---|---|---|
| | Example | Example | Example | Example | Example | Example |
| g/100 mL | 24 | 25 | 26 | 27 | 10 | 11 |
| All-trans form | 0.05 | 0.05 | — | — | 0.05 | — |
| All-trans form: | — | — | 0.05 | 0.05 | — | 0.05 |
| 5Z-mono-cis form | | | | | | |
| weight ratio (6:4) | | | | | | |
| Sodium dihydrogen | 2.00 | 0.30 | 2.00 | 0.30 | — | — |
| phosphate dihydrate | | | | | | |
| Disodium hydrogen | 0.40 | 3.20 | 0.40 | 3.20 | — | — |
| phosphate | | | | | | |
| dodecahydrate | | | | | | |
| Boric acid | — | — | — | — | 1.400 | 1.400 |
| Borax | — | — | — | — | 0.300 | 0.300 |
| POE castor oil | 0.02 | 0.02 | 0.02 | 0.02 | 0.02 | 0.02 |
| Polysorbate 80 | 0.50 | 0.50 | 0.50 | 0.50 | 0.50 | 0.50 |
| Dibutylhydroxytoluene | 0.005 | 0.005 | 0.005 | 0.005 | 0.005 | 0.005 |
| Hydrochloric acid | q.s. | q.s. | q.s. | q.s. | q.s. | q.s. |
| Sodium hydroxide | q.s. | q.s. | q.s. | q.s. | q.s. | q.s. |
| Purified water | q.s. | q.s. | q.s. | q.s. | q.s. | q.s. |
| pH | 5.7 | 7.5 | 5.7 | 7.5 | 7.5 | 7.5 |
| Osmotic pressure | 270 | 260 | 270 | 260 | 240 | 240 |
| mOsm | | | | | | |
| Adsorption amount | 85.3 | 71.2 | 84.1 | 83.7 | 112.4 | 120.1 |
| (μg/lens) | | | | | | |
| Immersion for | 89.7 | 85.3 | 96.1 | 85.5 | 111.0 | 133.9 |
| 8 hours | | | | | | |
| Adsorption amount | 229.1 | 209.5 | 251.9 | 243.3 | 287.1 | 328.4 |
| (μg/lens) | | | | | | |
| Immersion for | 212.9 | 213.6 | 254.6 | 235.5 | 298.1 | 345.8 |
| 24 hours | | | | | | |

As is apparent from Tables 5 and 6, the adsorption of GGA to contact lenses was smaller in the eye drops containing the phosphate buffering agents than in the eye drops containing the borate buffering agents.

### INDUSTRIAL APPLICABILITY

The ophthalmic composition of the present invention is excellent in the stability of GGA and adsorption of GGA in the composition to a container wall and a contact lens is remarkably reduced, and therefore the ophthalmic composition is very useful in practice.

1. An ophthalmic composition comprising geranylgeranylacetone and a phosphate buffering agent.

2. The ophthalmic composition according to claim 1, whose pH is from 6 to 8.

3. The ophthalmic composition according to claim 1, wherein the phosphate buffering agent concentration expressed in terms of a corresponding anhydride is 0.001 to 10% by weight relative to the total amount of the composition.

4. The ophthalmic composition according to any of claim 1, wherein the phosphate buffering agent is at least one selected from the group consisting of phosphoric acid and an alkali metal phosphate.

5. The ophthalmic composition according to any of claim 1, wherein the geranylgeranylacetone content is 0.00001 to 10% by weight relative to the total amount of the composition.

6. A method for reducing the loss of the geranylgeranylacetone content of an ophthalmic composition,
the method comprising the step of adding a phosphate buffering agent to an ophthalmic composition being held by an ophthalmic container and comprising geranylgeranylacetone,
thereby reducing the loss of the geranylgeranylacetone content of the ophthalmic composition.

7. A method for reducing white turbidity due to geranylgeranylacetone during storage at low temperature,
the method comprising the step of adding a phosphate buffering agent to an ophthalmic composition comprising geranylgeranylacetone,
thereby reducing white turbidity due to geranylgeranylacetone during storage at low temperature.

8. A method for reducing adsorption of geranylgeranylacetone to a contact lens,
the method comprising the step of adding a phosphate buffering agent to an ophthalmic composition comprising geranylgeranylacetone,
thereby reducing adsorption of geranylgeranylacetone to a contact lens.

9. A method for stabilizing geranylgeranylacetone,
the method comprising the step of adding a phosphate buffering agent to an ophthalmic composition comprising geranylgeranylacetone,
thereby stabilizing geranylgeranylacetone.

10. A method for reducing adsorption of geranylgeranylacetone to a wall of an ophthalmic container,
the method comprising the step of adding a phosphate buffering agent to an ophthalmic composition being held by an ophthalmic container and comprising geranylgeranylacetone,
thereby reducing adsorption of geranylgeranylacetone to a wall of the ophthalmic container.
